(19) United States
(12) Patent Application Publication
(10) Pub. No.: US 2016/0375180 A1
(43) Pub. Date: Dec. 29, 2016

(54) METHOD FOR PRODUCING MEDICAL DEVICE AND MEDICAL DEVICE

(71) Applicant: TERUMO KABUSHIKI KAISHA, Tokyo (JP)

(72) Inventor: Takao ANZAI, Hiratsuka-city (JP)

(73) Assignee: TERUMO KABUSHIKI KAISHA, Tokyo (JP)

(21) Appl. No.: 15/260,829

(22) Filed: Sep. 9, 2016

Related U.S. Application Data
(63) Continuation of application No. PCT/JP2015/056714, filed on Mar. 6, 2015.

(30) Foreign Application Priority Data
Mar. 11, 2014 (JP) 2014-047695

(51) Int. Cl. A61L 33/00; A61L 33/06

(52) U.S. Cl. CPC A61L 33/0029; A61L 33/0005; A61L 33/0041; A61L 33/064; A61L 33/0094; A61L 2400/10; A61L 2300/42; A61L 2420/02

(57) ABSTRACT

Provided is a method for preparing a medical device which has a surface lubricating layer on a surface of a substrate and the surface of which exhibits lubricity and antithrombotic qualities when being wet. The method includes forming a coating layer that contains a hydrophilic polymer having at least one reactive functional group selected from the group consisting of an epoxy group, an acid chloride group, and an aldehyde group, and then applying onto the coating layer an antithrombotic material solution that contains an antithrombotic material having a functional group capable of binding to the hydrophilic polymer to form the surface lubricating layer. The concentration of the antithrombotic material in the antithrombotic material solution is more than 0 and less than 0.1 wt %.

### CROSS-REFERENCES TO RELATED APPLICATIONS

This application is a continuation of International Application No. PCT/JP2015/056714 filed on Mar. 6, 2015, and claims priority to Japanese Application No. 2014-047695 filed on Mar. 11, 2014, the entire content of both of which is incorporated herein by reference.

### TECHNICAL FIELD

The present invention generally relates to a method for producing a medical device and a medical device. More particularly, the present invention relates to a method for producing a medical device having excellent surface lubricity and antithrombotic qualities, and a medical device.

### BACKGROUND DISCUSSION

Medical devices to be inserted into a living body, such as catheters, guide wires, indwelling needles, and the like, are required to exhibit excellent lubricity (surface lubricity) in order to improve operability of an operator while reducing damage to tissues such as blood vessels. For this reason, methods of coating the surface of a substrate layer with a hydrophilic polymer having lubricity have been developed and put into practical use.

In the use of the medical device, a synthetic polymer material, which is foreign to the living body, is used while being brought into contact with a biological tissue or a body fluid such as blood. Accordingly, medical materials are required to have biocompatibility. Although biocompatibility required for medical materials varies depending on the purpose or usage thereof, the medical materials to be used as a material in contact with blood are required to have properties (antithrombotic qualities) of suppression of the blood coagulation system, suppression of adhesion-activation of platelets, and suppression of activation of complement systems.

Typically, imparting antithrombotic qualities to the medical device is carried out by a method of coating a substrate constituting the medical device with an antithrombotic material or a method of fixing the antithrombotic material to a surface of the substrate.

For example, a sulfonic acid-containing polymer such as poly-2-acrylamido-2-methyl-propanesulfonic acid (PAMPS) is known as an antithrombotic material which has excellent anticoagulation activity and sterilization resistance, and may improve safety and durability. As a medical device using the PAMPS, for example, JP-A-8-24327 discloses a medical device in which a 2-acrylamido-2-methyl-propanesulfonic acid (AMPS)-acrylic acid (AA) copolymer as an antithrombotic material is fixed to a coating layer formed by coating the surface of a substrate with a hydrophilic polymer.

### SUMMARY

The medical device disclosed in Japanese Application Publication No. 8-24327 exhibits excellent surface lubricity and antithrombotic qualities. However, a medical device brought into contact with blood may be exposed to an environment in which thrombus is easily formed depending on the blood composition, the state of the blood flow or the state of the blood vessel wall in some cases. Furthermore, under severe conditions in which thrombus is relatively easily formed as described above, the antithrombotic qualities of the medical device disclosed in Japanese Application Publication No. 8-24327 may not be sufficient in some cases. In particular, during a neuroendovascular therapy by a catheter, in order to suppress cerebral infarction due to formation of thrombus on the surface of the catheter and to improve the operability, medical devices having excellent surface lubricity and antithrombotic qualities are needed.

Disclosed here is a method for producing a medical device having excellent surface lubricity and antithrombotic qualities under severe conditions in which thrombus is easily formed, and a medical device.

As a result of intensive studies, the present inventors have found that an improved method for producing a medical device is provided by setting the concentration of an antithrombotic material in an antithrombotic material solution to be applied onto a coating layer that contains a hydrophilic polymer previously formed within a specific range.

An aspect of the method disclosed here involves the following.

A method for producing a medical device comprising a surface lubricating layer on a surface of a substrate, in which the surface lubricating layer exhibits lubricity and antithrombotic qualities when being wet, the method comprising: forming onto the substrate a coating layer that contains a hydrophilic polymer having at least one reactive functional group selected from the group consisting of an epoxy group, an acid chloride group, and an aldehyde group; and applying onto the coating layer an antithrombotic material solution that contains an antithrombotic material having a functional group that binds to the hydrophilic polymer to form the surface lubricating layer. The antithrombotic material in the antithrombotic material solution is present in a concentration more than 0 wt % and less than 0.1 wt %

The antithrombotic material can have a sulfonate group or a sulfate group.

The antithrombotic material can have a repeating unit derived from a monomer selected from the group consisting of 2-(meth)acrylamido-2-methyl-propanesulfonic acid, vinyl sulfate, allyl sulfate, styrenesulfonic acid, sulfoethyl (meth)acrylate, and sulfopropyl (meth)acrylate, or salts thereof;

The functional group configured to bind to the hydrophilic polymer is a carboxyl group, a hydroxyl group, or a thiol group;

The hydrophilic polymer can be obtained by copolymerizing a monomer having the reactive functional group with a hydrophilic monomer; and

According to another aspect, a medical device comprises: a surface lubricating layer on a surface of a substrate, the surface exhibiting lubricity and antithrombotic qualities when being wet; the surface lubricating layer containing a hydrophilic polymer having at least one reactive functional group selected from the group consisting of an epoxy group, an acid chloride group, and an aldehyde group, and an antithrombotic material having a functional group that binds to the hydrophilic polymer; the antithrombotic material being contained in an amount of more than 0 wt % and less than 0.06 wt % based on a total weight of the surface lubricating layer; and when blood having a heparin concentration of 0.2 units/mL is brought into contact with the surface lubricating layer and circulated, a platelet retention rate determined by Equation 1 below is 80% or more.

Platelet retention rate (%)=(Number of platelets in blood 2 hours after blood circulation)/(Number of platelets in blood before blood circulation)×100 (Equation 1)

### BRIEF DESCRIPTION OF THE DRAWINGS

FIG. 1 is an enlarged photograph illustrating a surface of a medical device produced in Example 1-1.

FIG. 2 is an enlarged photograph illustrating a surface of a medical device produced in Comparative Example 1-2.

### DETAILED DESCRIPTION

Set forth below is a detailed description of embodiments of a method for producing a medical device, including forming a coating layer that contains a hydrophilic polymer, and then imparting an antithrombotic material to the coating layer, and a medical device that contains a hydrophilic polymer and an antithrombotic material having a specific concentration, representing examples of the inventive method and medical product disclosed here.

The present invention is not limited to the following embodiments. Also, in the present specification, “X to Y” indicating a range means “X or more and Y or less”, and “weight” and “mass”, “wt %” and “mass %”, and “parts by weight” and “parts by mass” are treated as being synonymous with each other. Also, unless otherwise specifically described, operation, physical properties, and the like are measured under the conditions of room temperature (20 to 25° C.)/relative humidity of 40 to 50%.

A first aspect of the disclosure here provides a method for producing a medical device having a surface lubricating layer on a surface of a substrate, of which the surface exhibits lubricity and antithrombotic qualities when being wet, the method including forming a coating layer that contains a hydrophilic polymer (a) having at least one reactive functional group selected from the group consisting of an epoxy group, an acid chloride group, and an aldehyde group, and then applying onto the coating layer an antithrombotic material solution (B) that contains an antithrombotic material (b) having a functional group capable of binding to the hydrophilic polymer (a) so as to form the surface lubricating layer, in which a concentration of the antithrombotic material (b) in the antithrombotic material solution (B) is more than 0 wt % and less than 0.1 wt %.

Hereinafter, the method for producing a medical device according to one embodiment representing an example of the inventive method disclosed here will be described in detail.

### [Method for Producing Medical Device]

In the disclosed method for producing a medical device according to the disclosure here (hereinafter, also simply abbreviated as “production method”), surface lubricity (lubricity when being wet; hereinafter, “lubricity” is intended to mean “lubricity when being wet” unless otherwise specifically described) and antithrombotic qualities are imparted to the medical device by forming a layer to be coated on at least a part of a surface of a substrate. As described above, the production method disclosed here includes forming a surface lubricating layer having surface lubricity and antithrombotic qualities.

In the step of forming a surface lubricant as described above, specifically, a coating layer that contains a hydrophilic polymer (a) is formed, and then an antithrombotic material solution (B) that contains an antithrombotic material (b) is applied onto the coating layer so as to form the lubricating layer. That is, the antithrombotic material solution (B) is applied onto the coating layer so as to form a surface lubricating layer in which antithrombotic materials are present in the top layer of the hydrophilic polymer coating layer, that is, within a certain depth range from the surface.

Furthermore, the production method disclosed here is characterized in that as described above, the concentration of the antithrombotic material in the antithrombotic material solution used for forming the surface lubricating layer is more than 0 wt % and less than 0.1 wt %.

The present inventors have found that when the antithrombotic material is fixed to a surface of the substrate by using the antithrombotic material solution as described above, a medical device having excellent antithrombotic and surface lubricity may be obtained by setting the concentration of the solution that contains the antithrombotic material within a specific range.

Japanese Application Publication No. 8-24327 discloses that in the production of a medical device, an antithrombotic material solution is impregnated with a coating layer formed by coating a surface of a substrate with a hydrophilic polymer. Furthermore, according to the technology, it is possible to obtain a medical device having surface lubricity and antithrombotic qualities. However, even in the technology, thrombus is formed in some cases under severe conditions in which thrombus is easily formed, and accordingly, antithrombotic is required to be further improved. Thus, as a result of intensive studies in order to further improve antithrombotic qualities, the present inventors have found that by setting the concentration of the antithrombotic material solution used when antithrombotic qualities are imparted to the coating layer that contains a hydrophilic polymer within a suitable range, excellent antithrombotic qualities may be obtained, and excellent surface lubricity is also retained.

The technology described in Japanese Application Publication No. 8-24327 describes that the AMPS-AA copolymer solution as an antithrombotic material solution has a concentration of 2 wt %, but the present inventors have surprisingly found that antithrombotic qualities are significantly improved by using an antithrombotic material solution having a lower concentration than the concentration described in the above-noted publication.

It is believed that the improvement in the antithrombotic qualities results from the charge (electric charge) balance between a hydrophilic polymer constituting a surface lubricating layer and an antithrombotic material. Herein, it is believed that the hydrophilic polymer is cationic (having properties which may be positively charged) and the antithrombotic material is anionic (having properties which may be negatively charged). Moreover, without wishing to be bound by any particular theory, it is believed that the balance between these electric charges may be appropriately maintained to improve the antithrombotic qualities. Accordingly, by rather maintaining a low concentration (specifically, more than 0 wt % and less than 0.1 wt %) instead of maintaining an antithrombotic material solution at high concentration to contain a large amount of an antithrombotic material in the surface lubricating layer, it is easier to suitably maintain the charge balance between the hydrophilic polymer and the antithrombotic material in the top layer of a coating layer that contains a hydrophilic polymer, and as a result, it is believed that an effect of improving antithrombotic qualities may be obtained. Therefore, according to the disclosure here, provided are a method for producing a medical device which has particularly surface lubricity, and excellent antithrombotic qualities under severe conditions in which thrombus is easily formed, and a medical device.

The mechanism or theory described above is based on current belief and understanding, but the invention here is not limited to that theory or mechanism.

Hereinafter, the formation of a surface lubricating layer (in the present specification, also simply referred to as “a surface lubricating layer forming step”) will be specifically described.

The surface lubricating layer forming step includes (I) forming a coating layer that contains a hydrophilic polymer (a) on a surface of a substrate, and (II) applying a solution (B) that contains an antithrombotic material (b) onto the coating layer. That is, the surface lubricating layer forming step includes (I) a coating layer forming step and (II) an antithrombotic material applying step. Also, in the surface lubricating layer forming step, an additional step (III) of modifying the surface lubricating layer may be further performed. Hereinafter, each of steps (I) to (III) will be described.

(I) Coating Layer Forming Step

The coating layer forming step is performed for the purpose of coating a surface of a substrate with a hydrophilic polymer (a).

The term “coating” is defined to include not only a form in which the entire surface of a substrate is completely covered by a hydrophilic polymer (a), but also a form in which only a part of the surface of the substrate is covered by the hydrophilic polymer (a), that is, a form in which the hydrophilic polymer (a) is attached to or applied on only a part of the surface of the substrate.

Therefore, the method for forming a coating layer is not particularly limited, except that the hydrophilic polymer (a) which is used has at least one reactive functional group selected from the group consisting of an epoxy group, an acid chloride group, and an aldehyde group, and may be applied in the same manner as a known method, or the method may be appropriately modified and applied. In the present specification, the term “hydrophilic polymer” refers to a polymeric compound having a water absorption rate of 1 g or more when immersed in 100 g of a physiological saline at 25° C.

As the method for forming the coating layer, specifically exemplified is a method for forming a coating layer including: dissolving the hydrophilic polymer (a) in a solvent to prepare a polymer solution (A) (a lubricant coating agent, a coating solution), coating the polymer solution (A) onto a substrate to form an application layer, and then subjecting the application layer to drying and heating treatment to form or result in the coating layer. That is, in the method disclosed here by way of example, it is preferred that the coating layer forming method at least includes a solution coating step of coating a polymer solution (A) that contains a hydrophilic polymer (a) on a substrate and a heating step of performing a heat treatment on an application layer formed by the polymer solution (A). By such method, lubricity and durability may be imparted to the surface of a medical device.

Hereinafter, preferred aspects of a coating layer forming step (I) will be described in detail.

(Preparation of Hydrophilic Polymer Solution)

As described above, in order to apply a polymer solution (A) that contains a hydrophilic polymer (a) on the substrate in a coating layer forming step, the polymer solution (A) is first prepared.

The hydrophilic polymer (a) is used to impart surface lubricity to a medical device. Therefore, as the hydrophilic polymer (a), a hydrophilic polymer, which may absorb water when being wet (wetted) to exhibit surface lubricity, is used. That is, the hydrophilic polymer (a) exhibits surface lubricity by absorbing a physiological saline, a buffer, an aqueous solvent or a body fluid such as blood.

In order to firmly fix (coat) the hydrophilic polymer (a) onto a substrate, the hydrophilic polymer (a) has at least one reactive functional group selected from the group consisting of an epoxy group, an acid chloride group, and an aldehyde group. In the present specification, the term “reactive functional group” refers to a functional group which may be crosslinked with other monomers by a heat treatment, light irradiation, electron beam irradiation, radiation irradiation, plasma irradiation, and the like. Further, the hydrophilic polymer (a) is firmly fixed to a substrate by the reactive functional group. Therefore, from the viewpoint of reactivity (fixation) with a substrate, handleability, efficiency of crosslinking reactions, and the like, it is preferred that the hydrophilic polymer (a) has an epoxy group as the reactive functional group.

It is preferred that the hydrophilic polymer (a) which has a reactive functional group as described above is obtained by copolymerizing a monomer (hereinafter, also referred to as “a reactive monomer”) which has a reactive functional group (in the molecule) with a hydrophilic monomer.

The reactive monomer has at least one reactive functional group selected from the group consisting of an epoxy group, an acid chloride group, and an aldehyde group as described above, and these reactive functional groups may be present either alone or in a plurality thereof in the reactive monomer. Furthermore, when a plurality of reactive functional groups is present, the reactive functional groups may be the same as each other or two or more different reactive functional groups.

It is preferred that the reactive monomer used to produce the hydrophilic polymer (a) has a reactive functional group and exhibits more hydrophobicity than the hydrophilic monomer used at least during the production of the hydrophilic polymer (a) in a body fluid or an aqueous solvent.

Specific examples of the reactive monomer include a monomer having an epoxy group in the molecule such as glycidyl acrylate, glycidyl methacrylate (GMA), methyl glycidyl methacrylate and allyl glycidyl a monomer having an acid chloride group in the molecule such as (meth)acrylic acid chloride; a monomer having an aldehyde group in the molecule such as (meth)acrylaldehyde, croton aldehyde, acrolein and methacrolein; and the like.

Among them, as the monomer having a reactive functional group, preferred is a monomer having an epoxy group, and more preferred is glycidyl acrylate or glycidyl methacrylate, which promotes the reaction by heat, and the like and is also relatively easily handled. These reactive monomers may be used either alone or in combination of two or more thereof. That is, the reactive site of the hydrophilic polymer (a) in the disclosure here may be a homopolymer type composed of one reactive monomer alone, or a copolymer type composed of two or more of the reactive monomers. The form of a polymer when two or more reactive monomers are used may be a block copolymer or a random copolymer.

In addition, the hydrophilic monomer used to produce the hydrophilic polymer (a) is not particularly limited, but examples thereof include, for example, acrylamide or derivatives thereof, vinyl pyrrolidone, acrylic acid or methacrylic acid, and derivatives thereof, polyethylene glycol acrylate and derivatives thereof, monomers having sugar or phospholipids in a side chain thereof, water-soluble monomers such as anhydrous maleic acid, and the like. More specific examples thereof include acrylic acid, methacrylic acid, N-methylacrylamide, N,N-dimethylacrylamide, acrylamide, acryloylmorpholine, N,N-dimethylaminoethylacrylate, vinylpyrrolidone, 2-methacryloyloxyethylphosphorylcholine, 2-methacryloyloxyethyl-D-glycoside, 2-methacryloyloxyethyl-D-mannoside, vinyl methyl ether, 2-hydroxyethyl (meth)acrylate, 4-hydroxybutyl (meth)acrylate, 2-hydroxypropyl (meth)arylate, 2-hydroxybutyl (meth)acrylate, 6-hydroxyhexyl (meth)acrylate, 1,4-cyclohexanedimethanolmono(meth)acrylate, 1-chloro-2-hydroxypropyl (meth)acrylate, diethylene glycol mono(meth)acrylate, 1,6-hexanediol mono(meth)acrylate, pentaerythritol tri(meth)acrylate, dipentaerythritol penta(meth)acrylate, neopentyl glycol mono(meth)acrylate, trimethylolpropane di(meth)acrylate, trimethylolethane di(meth)acrylate, 2-hydroxy-3-phenyloxypropyl (meth)acrylate, 4-hydroxycyclohexyl (meth)acrylate, 2-hydroxy-3-phenyloxy (meth)acrylate, 4-hydroxycyclohexyl (meth)acrylate, cyclohexanedimethanol mono(meth)acrylate, poly(ethylene glycol)methyletheracrylate, and poly(ethylene glycol)methylethermethacrylate. From the viewpoint of ease in synthesis or operability, N,N-dimethylacrylamide, acrylamide, acrylic acid, methacrylic acid, N,N-dimethylaminoethylacrylate, 2-hydroxyethylmethacrylate, and vinylpyrrolidone are preferred, N,N-dimethylacrylamide and N,N-dimethylaminoethylacrylate are more preferred, and N,N-dimethylacrylamide is particularly preferred. These hydrophilic monomers may be used either alone or in combination of two or more thereof. That is, the hydrophilic site of the hydrophilic polymer (a) disclosed here may be a homopolymer type composed of one hydrophilic monomer alone, or a copolymer type composed of two or more of the hydrophilic monomers. Therefore, it is preferred that the hydrophilic site is derived from one or more selected from the group consisting of the aforementioned hydrophilic monomers. The form of a polymer when two or more hydrophilic monomers are used may be a block copolymer or a random copolymer.

In order to exhibit excellent surface lubricity, the hydrophilic polymer (a) is preferably a polymer which is obtained by copolymerizing a reactive monomer with a hydrophilic monomer and has a reactive functional group capable of being crosslinked, and more preferably a block copolymer which has a block formed of a monomer having a reactive functional group and a block formed of a hydrophilic monomer. From the block copolymer, it is possible to obtain an excellent result in view of strength or lubricity of the surface lubricating layer.

Also, in more preferred exemplary embodiments disclosed here, the hydrophilic polymer (a) is a block copolymer that has a reactive site using a monomer having an epoxy group as at least one constituent unit and a hydrophilic site using a monomer which has a hydrophilic monomer as at least one constituent unit. The epoxy group, which is a reactive functional group, may be reacted with an adjacent epoxy group to allow an adjacent hydrophilic polymer to form a crosslinking structure, thereby increasing strength of the surface lubricating layer. Furthermore, the substrate may be bound to an epoxy group to suppress or prevent the coating layer from being peeled off from the substrate.

The ratio of the hydrophilic monomer and the reactive monomer in the hydrophilic polymer (a) is not particularly limited. In consideration of excellent lubricity, strength of films, firm binding property with a substrate layer, and the like, the molar ratio of the hydrophilic monomer and the reactive monomer is preferably the hydrophilic monomer:the reactive monomer=1:1 to 100:1, more preferably 5:1 to 80:1, and even more preferably 10:1 to 50:1. Within the ratios, the ratio of hydrophilic site and reactive site in the hydrophilic polymer may be maintained within a good range.

The method for producing the hydrophilic polymer (a) according to the disclosure here is not particularly limited, and the hydrophilic polymer (a) may be prepared by applying a conventionally known polymerization method in the related art, such as, for example, a living radical polymerization method, a polymerization method using a macroinitiator, and a polycondensation method. Among them, the living radical polymerization method or the polymerization method using a macroinitiator is preferably used, in that it is easy to control the molecular weight and molecular weight distribution of a site (reactive domain) derived from the reactive monomer and a site (hydrophilic domain) derived from the hydrophilic monomer. The living radical polymerization method is not particularly limited, but may be applied in the same manner as the methods described in, for example, Japanese Application Publication No. 11-263819, Japanese Application Publication No. 2002-145971, and Japanese Application Publication No. 2006-316169, and the like or may be applied by appropriately modifying the methods. Further, in the polymerization method using a macroinitiator, a block copolymer having a hydrophilic site and a reactive site may be manufactured by manufacturing a macroinitiator which has a reactive functional group and a radical polymerizable group such as a peroxide group, and then polymerizing the macroinitiator with a hydrophilic monomer.

In addition, in the polymerization of the block copolymer, a known method such as bulk polymerization, suspension polymerization, emulsion polymerization and solution polymerization may be used. A solvent appropriately used in the polymerization is not particularly limited, but it is possible to use, for example, an aliphatic organic solvent such as n-hexane, n-heptane, n-octane, n-decane, cyclohexane, methylcyclohexane, and liquid paraffin, an ether-based solvent such as tetrahydrofuran and dioxane, an aromatic organic solvent such as toluene and xylene, a halogen-based organic solvent such as 1,2-dichloroethane and chlorobenzene, and a polar non-protonic organic solvent such as N,N-dimethylformamide and dimethylsulfoxide (DMSO). The solvents may also be used either alone or in mixture of two or more thereof. The concentration of monomers in a polymerization solvent (concentration of a total weight of a hydrophilic monomer and a reactive monomer) is preferably 5 to 90 wt %, more preferably 8 to 80 wt %, and particularly preferably 10 to 50 wt %.

In order to obtain a block copolymer having a desired property, the polymerization temperature is preferably 50 to 100° C., more preferably 55 to 90° C., even more preferably 60 to 85° C., and particularly preferably 65° C. or more and less than 80° C.

Also, the polymerization time is preferably 1 to 24 hours, and more preferably 3 to 12 hours.

The hydrophilic polymer (a) obtained in the same manner as described above exhibits lubricity by wetting (absorbing water), thereby reducing friction resistance between a medical device and an inner wall of a living body. Moreover, in order to coat a surface of a substrate with the hydrophilic polymer (a), a polymer solution (A) of a hydrophilic polymer (a) is prepared using a suitable solvent.

A solvent used in the preparation of the polymer solution (A) is not particularly limited as long as the solvent may dissolve the hydrophilic polymer (a). Specific examples of the solvent include water, alcohols such as methanol, ethanol, isopropanol, and ethylene glycol, ketones such as acetone and methyl ethyl ketone, esters such as ethyl acetate, halides such as chloroform, olefins such as hexane, ethers such as tetrahydrofuran and butyl ether, aromatics such as benzene and toluene, amides such as N,N-dimethylformamide (DMF), and the like, but are not limited thereto at all. These solvents may be used either alone or in combination of two or more thereof.

The concentration of the hydrophilic polymer (a) in the polymer solution (A) is not particularly limited. From the viewpoint that applicability and desired effects (lubricity and durability) may be obtained, the concentration of the hydrophilic polymer (a) in the coating solution is 0.01 wt % to 20 wt %, more preferably 0.05 wt % to 15 wt %, and even more preferably 0.1 wt % to 10 wt %. When the concentration of the hydrophilic polymer (a) is within the above range, the lubricity and durability of a surface lubricating layer to be obtained may be sufficiently exhibited. Further, a surface lubricating layer with a desired uniform thickness may be relatively easily obtained by a one-time coating application within the above range, and the concentration is preferred in view of operability (for example, easy coatability) and production efficiency. However, even if the concentration is out of the aforementioned range, the concentration may be sufficiently used as long as the range of the concentration does not affect operational effects.

### (Application of Hydrophilic Polymer Solution)

In the coating layer forming step, the polymer solution (A) of the hydrophilic polymer (a) prepared as described above is subsequently applied on a surface of a substrate. In the present specification, the term “the surface of the substrate” refers to a substrate surface facing a biological tissue or a body fluid such as blood.

In the method and product disclosed here, the substrate constitutes a medical device. The material forming or constituting the substrate is not particularly limited, but it is preferred that at least the surface thereof is composed of a polymer material. Herein, the phrase “at least the surface thereof is composed of a polymer material” for the substrate means that at least the surface of the substrate may be composed of a polymer material, and is not limited to the entire substrate (all of the substrate) being composed (formed) of a polymer material. Therefore, in the context of the inventive method and medical device disclosed here, the substrate can also include a polymer material which is more flexible than a reinforced material such as a metal material and which is coated onto a surface of a core portion of a substrate formed of a relatively hard reinforced material such as a metal material and a ceramic material by a suitable method (a conventionally known method such as immersion (dipping), atomizing (spray), and application printing). Therefore, the substrate may have a multi-layer structure in which the core portion of the substrate is formed to have multilayers (multiple layers) by laminating different materials thereon as multilayers, or may have a structure (composite) in which members formed of different material are placed together and connected with each other for each part of the medical device. In addition, another intermediate layer may be additionally formed between the core portion of the substrate and the surface polymer layer. Furthermore, the surface polymer layer may also have a multi-layer structure in which the surface polymer layer is formed to have multilayers by laminating different materials thereon as multilayers, or may have a structure (composite) in which members formed of different material are placed together and connected with each other for each part of the medical device.

In the aforementioned form, a material, which may be used for the core portion of the substrate, is not particularly limited, and a reinforced material, which may sufficiently exhibit functions as a core portion of an optimal substrate, may be appropriately selected depending on the use of the medical device as a catheter, a guide wire, and an indwelling needle. Examples thereof include, for example, various stainless steels (SUS) such as SUS304, SUS316L, SUS420J2, and SUS630, various metal materials such as gold, platinum, silver, copper, nickel, cobalt, titanium, iron, aluminum, tin, and alloys thereof such as a nickel-titanium alloy, a cobalt-chromium alloy, and a zinc-tungsten alloy, inorganic materials such as various ceramic materials, and furthermore a metal-ceramic composite, and the like, but the examples are not limited thereto at all.

Further, the polymer material, which may be used for the substrate defining the surface to which the polymer layer is applied, is not particularly limited, and examples thereof include, for example, polyamide resins such as Nylon 6, Nylon 11, Nylon 12, and Nylon 66 (all registered trade names), polyethylene resins such as linear low-density polyethylene (LLDPE), low-density polyethylene (LDPE), and high-density polyethylene (HDPE), or polyolefin resins such as polypropylene resins, epoxy resins, urethane resins, diallylphthalate resins (allyl resins), polycarbonate resins, fluorine resins, amino resins (urea resins, melamine resins, and benzoguanamine resins), polyester resins, styrol resins, acrylic resins, polyacetal resins, vinyl acetate resins, phenolic resins, polyvinyl chloride (PVC) resins, silicone resins (silicon resins), and the like. These polymer materials may be used either alone or in combination of two or more thereof. In the polymer material, optimal polymer materials may be appropriately selected as a polymer substrate depending on the type of medical device such as a catheter, a guide wire, and an indwelling needle for usage.

In addition, the material, which may be used for the intermediate layer, is not particularly limited, and may be appropriately selected depending on the use thereof. Examples thereof include, for example, various metal materials, various ceramic material, and furthermore, organic-inorganic composites, and the like, but the examples are not limited thereto at all.

Herein, when a copolymer is desired to be firmly fixed by swelling a substrate with a solvent, it is preferred that a material forming the substance can be well swollen by a solvent for the polymer solution (A).

A method for applying the polymer solution (A) onto a surface of a substrate is not particularly limited, and it is possible to apply a conventionally known method in the related art, such as an application printing method, an immersion method (a dipping method, a dip coating method), an atomizing method (a spray method), a spin coating method, and a mixed solution impregnation sponge coating method. Among them, it is preferred to use an immersion method (a dipping method, a dip coating method). That is, according to the disclosure here, it is preferred that the coating layer is formed by immersing a substrate into a polymer solution (A) that contains a hydrophilic polymer (a).

The thickness (thickness of the application layer) of the polymer solution (A) may be appropriately adjusted depending on the use of a medical device, and is not particularly limited, but the thickness (thickness of a dried film) of the coating layer is, for example, preferably 0.1 μm to 10 μm, more preferably 0.5 μm to 5 μm, even more preferably 1 μm to 5 μm, and particularly preferably 1 μm to 3 μm.

Furthermore, when a coating layer is formed on only a part of the substrate, the coating layer may be formed on a desired surface site of the substrate by immersing only a part of the substrate into the polymer solution (A) to coat the part of the substrate with the polymer solution (A).

When it is difficult to immerse only a part of the substrate in the polymer solution (A), it is appropriate to protect (cover, coat, and the like) a surface portion of the substrate, on which the coating layer does not need to be applied, with a suitable member or material (protective member or protective material) capable of being attached or detached, then immerse the substrate into the polymer solution (A) to coat the substrate with the polymer solution (A), and then remove the protective member (material) of the surface portion of the substrate, on which the coating layer does not need to be formed. The immersion condition in this case may also vary depending on the thickness of the coating layer formed, but it is preferred that the immersion condition is performed, for example, at 10 to 50° C. for 1 to 60 seconds.

For the coating layer to be more stably fixed to the surface of the substrate, the substrate may also be subjected to surface treatment before the polymer solution (A) is applied onto the surface of the substrate. Examples of the surface treatment method for the substrate include, for example, a method of irradiating active energy rays (electron beam, ultraviolet rays, X-rays, and the like), a method of using plasma discharge such as arc discharge, corona discharge and glow discharge, a method of applying high electrical field, a method of applying ultrasound vibration through a polarity liquid (water and the like), an ozone gas treatment method, and the like.

### (Drying and Heating (Fixation) Treatment)

In the coating layer forming step, a coating layer of a hydrophilic polymer (a) is formed on a surface of a substrate by applying a polymer solution (A) that contains a hydrophilic polymer (a) onto the substrate (carrying out immersion) as described above, and then drying the surface of the substrate. Furthermore, it is preferred that the substrate is immersed into the polymer solution (A) to coat the substrate, and then a heat treatment is additionally performed thereon as described below in detail. A surface lubricating layer may be firmly fixed on a desired surface site of the substrate by performing the heat treatment thereon to cause the reactive functional group of the hydrophilic polymer (a) to react therewith.

Herein, the heat treatment conditions (temperature, time, and the like) of the coating solution are not particularly limited as long as a coating layer that contains a hydrophilic polymer may be formed on the substrate. Specifically, the heating temperature is preferably 60° C. to 200° C., more preferably 65° C. to 160° C., and even more preferably 70° C. to 150° C. Also, the heating time is preferably 15 minutes to 24 hours, and more preferably 1 hour to 10 hours. Under these conditions, a crosslinking-reaction caused by the reactive functional group of the hydrophilic polymer (a) occurs, so that a firm surface lubricating layer may be formed without being easily peeled off from the substrate.

When an epoxy group is adopted as the reactive functional group included in the hydrophilic polymer (a), the epoxy group may be heated to be self-crosslinked, and in order to promote the crosslinking reaction, an epoxy reaction catalyst or a polyfunctional crosslinking agent capable of being reacted with the epoxy group may also be included in the polymer solution (A).

In addition, the pressure conditions during the heat treatment are not limited at all, and the treatment may be carried out under pressurized pressure or reduced pressure, in addition to under normal pressure (atmospheric pressure).

As a heat treatment apparatus (device), for example, an oven, a vacuum dryer, and the like may be used.

Furthermore, the coating layer that contains the hydrophilic polymer may also be laminated in two or more layers. Therefore, after the drying and heating treatment, additionally, a step of applying the polymer solution (A) may be repeatedly performed.

(II) Antithrombotic Material Applying Step

The antithrombotic material applying step is carried out for the purpose of forming a surface lubricating layer which has excellent antithrombotic qualities by imparting an antithrombotic material to the coating layer formed in Step (I). Herein, the term “surface lubricating layer” refers to a state where an antithrombotic material (b) is dispersed and fixed in or on the top layer of the coating layer including the hydrophilic polymer (a), and the surface lubricating layer includes the hydrophilic polymer (a) and the antithrombotic material (b).

In the method for producing a medical device as disclosed here, it is also one of the characteristics to form a coating layer that contains a hydrophilic polymer (a) as described above, and then impart an antithrombotic material (b) to the coating layer. That is, in the production method disclosed here, when a surface lubricating layer is formed, a coating layer that contains a hydrophilic polymer (a) is first formed, and then an antithrombotic material (b) is imparted to the coating layer, instead of applying the hydrophilic polymer (a) and the antithrombotic material (b) onto the substrate at one time. As described below in detail, the hydrophilic polymer (a) and the antithrombotic material (b) may be crosslinked with each other, so that when a solution in which these two coexist is prepared, there is concern in that most of the reactive functional groups included in the hydrophilic polymer (a) may be crosslinked with the antithrombotic material (b). In this case, a number of reactive functional group sufficient to firmly fix the hydrophilic polymer (a) to the surface of the substrate may not be secured. As a result, the coating layer may not be firmly fixed to the substrate, so that the durability of the surface lubricating layer deteriorates. In this regard, since a surface lubricating layer is formed by two steps that include the coating layer formation step and the antithrombotic material applying step, the reactive functional group of the hydrophilic polymer (a) may be first fixed to the substrate, the coating layer of the hydrophilic polymer may be formed by a crosslinking reaction, and then the other reactive functional groups may be crosslinked with the antithrombotic material (b). Therefore, much higher lubricity may be obtained from the surface lubricating layer because the remains of the hydrophilic polymer (a) are firmly fixed to the substrate. Further, since higher antithrombotic qualities are imparted to the antithrombotic material (b), it is possible to improve surface lubricity and antithrombotic qualities of the surface lubricating layer.

Moreover, for the production method disclosed here, an antithrombotic material solution (B) that contains an antithrombotic material (b) having a functional group capable of binding to a hydrophilic polymer (a) is applied onto the coating layer that contains the hydrophilic polymer (a) in the antithrombotic material applying step, and in this case, the concentration of the antithrombotic material (b) is more than 0 wt % and less than 0.1 wt %.

Hereinafter, the antithrombotic material applying step, which is a characteristic factor of the method and medical device disclosed here, will be described in detail.

### (Preparation of Antithrombotic Material Solution)

In the antithrombotic material applying step, in order to apply an antithrombotic material solution (B) that contains an antithrombotic material (b) onto the coating layer formed as described above, an antithrombotic material solution (B) is first prepared.

The antithrombotic material (b) has a functional group capable of binding to or configured to bind to the hydrophilic polymer (a) (in the present specification, also simply referred to as “a binding functional group”), and exhibits antithrombotic qualities while being bound to the hydrophilic polymer (a) by the functional group.

In this case, the binding functional group of the antithrombotic material (b) refers to a functional group which may be crosslinked with the functional group (preferably a reactive functional group of the hydrophilic polymer (a)) in the hydrophilic polymer (a) by a heat treatment, light irradiation, electron beam irradiation, radiation irradiation, plasma irradiation, and the like. Examples of the functional group include a carboxyl group (—COOH), a hydroxyl group (—OH), a thiol group (—SH), an amino group (—NH2), an amide group (—C(═O)NH—), a carboxylic acid anhydride group (—C(═O)—O—C(═O)—), and the like. These functional groups are easily crosslinked with the reactive functional group contained in the above-described hydrophilic polymer (a), and as a result, are suitable because the antithrombotic material (b) is firmly fixed to the coating layer that contains the hydrophilic polymer (a).

Among the functional groups, the binding functional group is particularly preferred as long as the binding functional group is a functional group which is negatively charged. When the antithrombotic material (b) includes a negatively charged functional group, a suitable charge balance with a positively charged functional group of the hydrophilic polymer (a) may be easily established, so that antithrombotic qualities may be improved. Further, even in the functional groups which are negatively charged, the binding functional group of the antithrombotic material (b) is preferably a carboxyl group, a hydroxyl group, or a thiol group from the viewpoint of handleability, efficiency of crosslinking reactions, and the like. Furthermore, the binding functional group is particularly preferably a carboxyl group, in that the carboxyl group suitably maintains a charge balance with the hydrophilic polymer (a) to particularly improve antithrombotic qualities.

Also, it is preferred that the antithrombotic material (b) has a sulfonate group (—SO3H) or a sulfate group (—OSO3H) in order to exhibit excellent antithrombotic qualities. The antithrombotic material (b) having these functional groups exhibit particularly excellent antithrombotic qualities due to negative charges derived from these functional groups. Furthermore, it is particularly preferred to have a sulfonate group from the viewpoint of the improvement of antithrombotic qualities. Further, the antithrombotic material (b) may include one of the functional group or may include both of these functional groups.

The antithrombotic material (b) having the binding functional group and a sulfonate group or a sulfate group is preferably an antithrombotic material obtained by copolymerizing a monomer having a binding functional group in the molecule (hereinafter, also referred to as “a binding monomer”) with a monomer having a sulfonate group or a sulfate group (hereinafter, also referred to as “an antithrombotic monomer”).

In this case, it is preferred that the binding monomer used has at least one reactive functional group selected from the aforementioned binding functional groups, and these binding functional groups may be present either alone or in a plurality thereof in the binding monomer. In addition, when a plurality of binding functional groups are present, the binding functional groups may be the same as each other or two or more different binding functional groups.

The binding monomer is not particularly limited. Examples thereof include acrylic acid, methacrylic acid, maleic acid, fumaric acid, glutaconic acid, itaconic acid, crotonic acid, sorbic acid, cinnamonic acid, N-(meth)acryloylglycine, N-(meth)acryloylasparaginic acid, N-(meth)acryloyl-5-aminosalicylic acid, 2-(meth)acryloyloxyethyl hydrogen succinate, 2-(meth)acryloyloxyethyl hydrogen phthalate, 2-(meth)acryloyloxyethyl hydrogen maleate, 6-(meth)acryloyloxyethylnaphthalene-1,2,6-tricarboxylic acid, O-(meth)acryloyltyrosine, N-(meth)acryloyltyrosine, N-(meth)acryloylphenylalanine, N-(meth) acryloyl-p-aminobenzoic acid, N-(meth)acryloyl-o-aminobenzoic acid, p-vinylbenzoic acid, 2-(meth)acryloyloxybenzoic acid, 3-(meth)acryloyloxybenzoic acid, 4-(meth)acryloyloxybenzoic acid, N-(meth)acryloyl-5-aminosalicylic acid, N-(meth)acryloyl-4-aminosalicylic acid and derivatives thereof; hydroxyethyl acrylate, hydroxyethyl methacrylate, and derivatives thereof, and the like.

The binding monomer (b) is preferably acrylic acid, methacrylic acid, and maleic acid, more preferably acrylic acid and methacrylic acid, and particularly preferably acrylic acid from the viewpoint of ease in synthesis or operability. These binding monomers may be used either alone or in combination of two or more thereof. That is, the binding site of the antithrombotic material (b) disclosed here may be a homopolymer type composed of one binding monomer alone, or a copolymer type composed of two or more of the binding monomers. When two or more binding monomers are used, the form of a polymer may be a block copolymer or a random copolymer.

In addition, the monomer (antithrombotic monomer) having a sulfonate group or a sulfate group used to produce the antithrombotic material (b) is not particularly limited, but examples thereof include 2-(meth)acrylamido-2-methyl-propanesulfonic acid (AMPS), vinyl sulfate, allyl sulfate, styrenesulfonic acid, sulfoethyl (meth)acrylate, sulfopropyl (meth)acrylate, and the like. The sulfonate group and the sulfate group in these monomers may form a salt, and examples of the salt include salts of inorganic cations or salts of organic cations. The salts of inorganic cations are preferably alkali metal salts and alkaline earth metal salts, and among them, sodium salts, potassium salts, and lithium salts are more preferred. The salts of organic cations are preferably ammonium salts.

As preferred exemplary embodiments disclosed here, the antithrombotic monomer for producing the antithrombotic material (b) is preferably 2-(meth)acrylamido-2-methyl-propanesulfonic acid, vinyl sulfate, allyl sulfate, styrenesulfonic acid, sulfoethyl (meth)acrylate, and sulfopropyl (meth)acrylate, or salts thereof from the viewpoint of the improvement of compatibility with the hydrophilic polymer (a) or antithrombotic qualities, and the like. That is, it is preferred that the antithrombotic material (b) has a repeating unit derived from a monomer selected from the group consisting of 2-(meth)acrylamido-2-methyl-propanesulfonic acid, vinyl sulfate, allyl sulfate, styrenesulfonic acid, sulfoethyl (meth)acrylate, and sulfopropyl (meth)acrylate, or salts thereof.

Furthermore, the antithrombotic monomer used to produce the antithrombotic material (b) is preferably 2-(meth)acrylamido-2-methyl-propanesulfonic acid, vinyl sulfate, o-, m-, and p-styrenesulfonic acids, or salts thereof in view of antithrombotic qualities, anticoagulation activity, and blood compatibility, and more preferably 2-(meth)acrylamido-2-methyl-propanesulfonic acid or salts thereof, which may exhibit more excellent anticoagulation activity.

The antithrombotic monomer has water solubility or water swellability, and thus is soluble in various aqueous solvents. Accordingly, in an aqueous solvent such as body fluid, a copolymer, that contains a constituent unit derived from a sulfonate group-containing monomer, may be swollen on the surface of the coating layer, and thus may form an interface (an outermost layer) with an aqueous solvent, thereby effectively exhibiting antithrombotic qualities, anticoagulation activity, and blood compatibility. The aforementioned antithrombotic monomers may be used either alone or in combination of two or more thereof.

Herein, the terminal of the antithrombotic material (b) is not particularly limited, is appropriately defined depending on the kind of raw material used, and is typically a hydrogen atom. Also, the structure of the antithrombotic material (b) is also not particularly limited, and may be any one of a random copolymer, an alternating copolymer, a periodic copolymer, and a block copolymer. However, the random copolymer where cross-linking points are dispersed is preferred from the viewpoint of the improvement of the film strength (strength of the cross-linked structure) after being imparted to the coating layer.

In addition, in more preferred exemplary embodiments disclosed here, the antithrombotic material (b) is a random copolymer having a binding site that contains a monomer having a carboxyl group as at least one constituent unit and an antithrombotic site which includes an antithrombotic monomer having a sulfonate group or a sulfate group as at least one constituent unit. By reacting a carboxyl group which is a binding functional group with a reactive functional group in the hydrophilic polymer (a), the antithrombotic material (b) may form a crosslinking structure with the hydrophilic polymer (a), and may increase the strength of the surface lubricating layer. Furthermore, a carboxyl group, a sulfonate group, and a sulfuric acid may be negatively charged, and thus easily establish a suitable balance with electric charges of the above-described hydrophilic polymer (a), and as a result, antithrombotic qualities may be improved.

The antithrombotic material (b) according to the embodiments disclosed here by way of example has a weight average molecular weight of preferably 100,000 to 10,000,000. When the weight average molecular weight is in the above range, the weight average molecular weight is preferred in view of solubility. The weight average molecular weight of the copolymer is more preferably 1,000,000 to 10,000,000 in view of ease in preparation of the coating solution. Also, the antithrombotic material (b) has a number average molecular weight of preferably 10,000 to 10,000,000. When the number average molecular weight is in the above range, the number average molecular weight is preferred in view of solubility. The number average molecular weight of the copolymer is more preferably 100,000 to 1,000,000 in view of ease in preparation of the coating solution. In the disclosure here, the term “weight average molecular weight” adopts values measured by gel permeation chromatography (GPC) using polystyrene as a standard material and tetrahydrofuran (THF) as a mobile phase. Also, the number average molecular weight adopts values measured in the same manner as described above. Note that the weight average molecular weight and the number average molecular weight were measured by the same method even in the following Examples and Comparative Examples.

The ratio of the binding monomer and the antithrombotic monomer in the antithrombotic material (b) is not particularly limited. In consideration of excellent lubricity, strength of films, firm binding property with a substrate layer, and the like, the molar ratio of the binding monomer and the antithrombotic monomer is preferably the binding monomer:the antithrombotic monomer=1:1 to 1:100, more preferably 1:3 to 1:80, and even more preferably 1:5 to 1:50. When the ratio is within the above range, it is possible to obtain an antithrombotic material (b) which exhibits high antithrombotic qualities and is firmly bound to the reactive site of the hydrophilic polymer (a).

The method for producing the antithrombotic material (b) is not particularly limited. Typically, a method of copolymerizing the binding monomer and one or two or more of the antithrombotic monomers by stirring and heating the monomers together with the polymerization initiator in a polymerization solvent is used.

The method for producing the antithrombotic material (b) according to the disclosure here is not particularly limited, and for example, a known polymerization method such as radical polymerization, anionic polymerization, and cationic polymerization may be adopted, and preferably, the radical polymerization, by which the antithrombotic material (b) is easily produced, is used.

The polymerization initiator is not particularly limited, and a known polymerization initiator may be used. Preferably, a redox-based polymerization initiator is used in that the polymerization stability is excellent, and specifically, examples thereof include a system obtained by combining a reducing agent such as sodium sulfite, sodium hydrogen sulfite, and ascorbic acid with an oxidizing agent such as persulfate such as potassium persulfate (KPS), sodium persulfate, and ammonium persulfate; and a peroxide such as hydrogen peroxide, t-butyl peroxide, and methyl ethyl ketone peroxide.

The blending amount of the polymerization initiator is preferably 0.0001% by mole to 5% by mole based on the monomer (the total amount of the binding monomer and the antithrombotic monomer is 100% by mole).

It is preferred that the polymerization temperature is set to 30° C. to 100° C. in view of controlling the molecular weight. The polymerization is usually carried out for 30 minutes to 24 hours. The polymerization solvent is preferably an aqueous solvent such as water, alcohol, and polyethylene glycol, and particularly preferably water. These polymerization solvents may be used either alone or in combination of two or more thereof. The concentration (concentration of the solid content) of the monomer in the polymerization solvent is typically 3 wt % to 80 wt %, and preferably 5 wt % to 60 wt %. The monomer concentration based on the polymerization solvent indicates the concentration of the total weight of the binding monomer and the antithrombotic monomer.

Furthermore, during the copolymerization, a chain-transfer agent, a polymerization rate modifier, a surfactant, and other additives may be appropriately used, if necessary.

It is preferred that the antithrombotic material (b) after the copolymerization is purified by a general purification method such as a re-precipitation method, a dialysis method, an ultrafiltration method, and an extraction method. Also, the antithrombotic material (b) after the purification may also be dried by any method such as freeze drying, vacuum drying, spray drying or heat drying, but freeze drying or vacuum drying is preferred from the viewpoint that physical properties of the antithrombotic material (b) are minimally affected.

The antithrombotic material (b) obtained as described above is fixed to the coating layer that contains the hydrophilic polymer (a), and accordingly, the medical device disclosed here exhibits antithrombotic qualities. Since the antithrombotic material (b) is coated (fixed) onto the coating layer, an antithrombotic material solution (B) of the antithrombotic material (b) is prepared by using a suitable solvent.

A solvent used in the preparation of the antithrombotic material solution (B) is not particularly limited as long as the solvent may dissolve the antithrombotic material (b). Specific examples thereof include water, alcohols such as methanol, ethanol, isopropanol, and ethylene glycol, ketones such as acetone and methyl ethyl ketone, esters such as ethyl acetate, halides such as chloroform, olefins such as hexane, ethers such as tetrahydrofuran and butyl ether, aromatics such as benzene and toluene, amides such as N,N-dimethylformamide (DMF), and the like. In consideration of handleability or affinity for the hydrophilic polymer (a), water and alcohols are preferred, and water is particularly preferred. These solvents may be used either alone or in combination of two or more thereof.

The concentration of the antithrombotic material (b) in the antithrombotic material solution (B) is more than 0 wt % and less than 0.1 wt %. By setting the concentration within the above range, the coating layer (surface lubricating layer) coated with the antithrombotic material solution (B) exhibits excellent surface lubricity and excellent antithrombotic qualities.

Furthermore, from the viewpoint that desired effects (surface lubricity and antithrombotic) are further improved, the concentration of the antithrombotic material (b) in the coating solution is 0.005 wt % to 0.08 wt %, more preferably 0.008 wt % to 0.06 wt %, and particularly preferably 0.01 wt % to 0.05 wt %. In addition, by setting the concentration within the above range, a desired amount (that is, an amount suitable for obtaining high antithrombotic qualities) of the antithrombotic material (b) may be imparted to the coating layer by coating the material at one time, and a surface lubricating layer having a uniform thickness may be easily obtained. Furthermore, the above-described concentration range is preferred in view of operability (for example, ease in coating) and production efficiency.

### (Application of Antithrombotic Material Solution)

In the antithrombotic material applying step, an antithrombotic material solution (B) of the antithrombotic material (b) prepared as described above is subsequently applied onto the surface of the coating layer.

A method for applying the antithrombotic material solution (B) onto a surface of a substrate is not particularly limited, and it is possible to apply a conventionally known method in the related art, such as an application printing method, an immersion method (a dipping method, a dip coating method), an atomizing method (a spray method), a spin coating method, and a mixed solution impregnation sponge coating method. Among them, from the viewpoint that the antithrombotic material (b) is easily impregnated (permeated) into the coating layer that contains the hydrophilic polymer (a), it is preferred to use an immersion method (a dipping method, a dip coating method). That is, according to the embodiments of the method disclosed here by way of example, it is preferred that the surface lubricating layer is formed by immersing the coating layer into the antithrombotic material solution (B) that contains the antithrombotic material (b).

The antithrombotic material (b) is fixed to the surface of a medical device by applying the antithrombotic material (b) onto a coating layer. The term “fixation” referred to herein may be in a state where the antithrombotic material (b) is fixed while not being easily released (peeled off) from the coating layer, may be in a state where the antithrombotic material (b) is accumulated on the surface of the coating layer, and may be in a state where the antithrombotic material (b) is dispersed (impregnated) over a certain depth with respect to the surface of the coating layer. Herein, in consideration of the balance between surface lubricity and antithrombotic qualities, it is preferred that the antithrombotic material (b) is fixed while being dispersed over a certain depth with respect to the surface of the coating layer that contains the aforementioned hydrophilic polymer (a). In this case, it is preferred that the antithrombotic material (b) is dispersed in a range of 0.5 μm or less from the outermost surface of the coating layer. Also, it is more suitable for the antithrombotic material (b) to be dispersed in a range of 0.1 μm or less. This is because excellent antithrombotic qualities may be exhibited without impairing surface lubricity by dispersing the antithrombotic material (b) in a range of 0.5 μm or less from the outermost surface of the coating layer. That is, in the disclosure here, it is preferred to adopt a form where the antithrombotic material (b) is loaded only around the surface of the coating layer, rather than a form where the antithrombotic material (b) is dispersed throughout the coating layer. By partially loading the antithrombotic material (b) in the coating layer as described above, high antithrombotic qualities may be obtained without impairing the surface lubricity due to the hydrophilic polymer (a). Further, in the present specification, the term “outermost surface of the coating layer” refers to a surface facing a biological tissue or a body fluid such as blood in the coating layer.

Moreover, the configuration may be easily implemented by applying the antithrombotic material solution (B) onto the coating layer, in which the concentration of the antithrombotic material (b) is more than 0 wt % and less than 0.1 wt %, which is a relatively low value. The application (immersion) of the antithrombotic material solution (B) may be carried out one time or several times, but by setting the concentration of the antithrombotic material solution (B) within the above range, a medical device which includes excellent antithrombotic qualities and surface lubricity may be produced by application (immersion) one time. Therefore, the imparting of the antithrombotic material (b) during the production of a medical device is simplified, and accordingly, productivity is also excellent.

As described above, in preferred exemplary embodiments representing examples of the inventive method and medical device disclosed here, the application of the antithrombotic material solution (B) onto the coating layer previously formed on the substrate is carried out by the immersion method. In this case, the temperature is not particularly limited, but is preferably 10° C. to 50° C., and more preferably 15° C. to 40° C. By setting the temperature within the above range, the antithrombotic material (b) is efficiently loaded in the coating layer. Further, the immersion time is not particularly limited, but is preferably 1 minute to 1 hour, and more preferably 3 minutes to 30 minutes. By setting the immersion time to 1 minute or more, a sufficient amount of the antithrombotic material (b) may be loaded in the coating layer. Also, it is preferred to set the immersion time to 1 hour or less in view of productivity and simultaneously because it is possible to prevent the hydrophilic polymer (a) included in the substrate or coating layer from deteriorating.

### (Drying and Heating (Fixation) Treatment)

In the antithrombotic material applying step, a surface lubricating layer is formed on the surface of a substrate by applying the antithrombotic material solution (B) onto the coating layer (carrying out immersion) as described above, and then drying the surface of the coating layer. Furthermore, it is preferred that the coating layer is immersed into the antithrombotic material solution (B) to coat the coating layer, and then a heat treatment is additionally performed thereon as described below in detail. A firmer surface lubricating layer may be formed by forming the heat treatment thereon to promote a reaction between the reactive functional group of the hydrophilic polymer (a) and the binding functional group of the antithrombotic material (b).

Herein, the heat treatment conditions (temperature, time, and the like) of the coating layer after the application of the antithrombotic material (b) are not particularly limited as long as the conditions may promote the crosslinking reaction between the hydrophilic polymer (a) and the antithrombotic material (b) to be included in the coating layer without causing deterioration in the substrate or the coating layer. Specifically, the heating temperature is preferably 60° C. to 200° C., more preferably 65° C. to 160° C., and even more preferably 70° C. to 150° C. Also, the heating time is preferably 15 minutes to 24 hours, and more preferably 1 hour to 10 hours. Under these conditions, a crosslinking-reaction between the hydrophilic polymer (a) and the antithrombotic material (b) occurs, so that it is possible to form a firm surface lubricating layer from which the antithrombotic material is minimally released.

Also, the pressure conditions during the heat treatment are not limited at all, and the treatment may be carried out under pressurized pressure or reduced pressure, in addition to under normal pressure (atmospheric pressure).

As a heat treatment apparatus (device), for example, an oven, a vacuum dryer, and the like may be used.

(III) Other Steps

As described above, after the steps (I) and (II) are carried out, a step of washing and drying the surface lubricating layer or a step of modifying the surface lubricating layer may additionally be carried out.

In the step of washing the surface lubricating layer, the washing method is not particularly limited, but may be a method of immersing the surface lubricating layer into a washing solvent and extracting the surface lubricating layer, a method of performing shower with a washing solvent, or a combination thereof. In this case, the washing solvent is not particularly limited as long as the solvent does not dissolve the surface lubricating layer, but water or a hydrophilic solvent is preferably used. Examples of the hydrophilic solvent include methanol, ethanol, isopropyl alcohol, ethylene glycol, diethylene glycol, propylene glycol, and the like. Water and these solvents may be used either alone or in mixture of two or more thereof. Further, it is preferred that the drying step is subsequently carried out. The drying method is not particularly limited, and a conventionally known method in the related art may be used.

As the step of modifying the surface lubricating layer, for example, a step of further adding other antithrombotic materials may also be carried out for the purpose of improving antithrombotic qualities with respect to the surface lubricating layer. As the antithrombotic material which may be used in this case, it is possible to use a drug which suppresses thrombus from being produced or a material which dissolves thrombus, and examples thereof include natural and synthetic materials such as an anticoagulant, antiplatelet agent, a fibrinolysis promoting agent, and the like. Examples thereof include various coagulation-type protease inhibitors such as heparin, low-molecular heparin, dermatan sulfate, heparan sulfate, activated protein C, hirudin, aspirin, thrombomodulin, DHG, plasminogen activator, streptokinase, urokinase, aprotinin, nafamostat mesilate (FUT), and gabexate mesilate (FOY), but the examples are not limited thereto.

The method for imparting the antithrombotic material to the surface lubricating layer is not particularly limited, and a conventionally known method in the related art may be applied. Among them, it is preferred to use an immersion method (a dipping method, a dip coating method) from the viewpoint of operability. In this case, with respect to the method of preparing an application solution (coating solution) which includes the antithrombotic material and the method of application, a conventionally known method in the related art may be applied.

### [Medical Device]

According to the aforementioned production, provided is a medical material which has excellent surface lubricity and antithrombotic qualities. That is, another aspect of the disclosure here provides a medical device produced by the production method. Also, the medical device in preferred exemplary embodiments is a medical device which has a surface lubricating layer on a surface of a substrate and the surface of which exhibits lubricity and antithrombotic qualities when being wet, in which the surface lubricating layer includes a hydrophilic polymer (a) having at least one reactive functional group selected from the group consisting of an epoxy group, an acid chloride group, and an aldehyde group, and an antithrombotic material (b) having a functional group capable of binding to the hydrophilic polymer (a), and includes the antithrombotic material (b) in an amount of more than 0 wt % to less than 0.06 wt % based on a total weight of the surface lubricating layer (the total weight of the surface lubricating layer is set to 100 wt %), and when blood having a heparin concentration of 0.2 units/mL is brought into contact with the surface lubricating layer and circulated, a platelet retention rate obtained by Equation 1 below is 80% or more. One heparin unit is an amount to suppress 1 mL of blood from being coagulated for 1 hour.

[Math. 2]

Platelet retention rate (%)=(Number of platelets in blood 2 hours after blood circulation)/(Number of platelets in blood before blood circulation)×100 (Equation 1)

As described above, in the medical device according to the disclosure here, the antithrombotic material (b) in the surface lubricating layer is included in an amount of more than 0 wt % and less than 0.06 wt % based on the total weight of the surface lubricating layer. Further, the weight ratio of the antithrombotic material (b) is more preferably 0.003 wt % to 0.04 wt %, and even more 0.006 wt % to 0.035 wt %. When the weight ratio of the antithrombotic material (b) is 0 wt %, the antithrombotic qualities are not exhibited, and meanwhile, when the ratio is 0.06 wt % or more, it becomes difficult for the charge balance between the hydrophilic polymer and the antithrombotic material to be suitably maintained, and as a result, the antithrombotic qualities deteriorate. The weight ratio of the antithrombotic material (b) adopts values measured by the method described in the Example. As described above, it is possible to obtain a medical device having excellent surface lubricity and antithrombotic qualities by setting the weight ratio of the antithrombotic material (b) to the hydrophilic polymer (a) within the above range.

In addition, the medical device according to the disclosure here also has the following properties. That is, in the medical device, after blood is circulated for a predetermined time so as to be in contact with the surface lubricating layer, and before blood is circulated, the number of platelets in the circulated blood is maintained at a predetermined ratio or more. More specifically, in the medical device disclosed by way of example here, the platelet retention rate obtained by Equation 1 is 80% or more when blood having a heparin concentration of 0.2 units/mL is circulated in room temperature while being brought into contact with the surface lubricating layer. When the value is within the above range, it is possible to effectively suppress thrombus from being formed under a severe condition in which thrombus is easily formed.

Herein, the evaluation will be described. In the evaluation, heparin as an anticoagulant is added to circulating blood to manufacture a blood sample having a heparin concentration of 0.2 units/mL, and the number of platelets in blood (platelet retention rate) is evaluated before and after the blood sample is circulated (distributed) for 2 hours while being brought into contact with a surface lubricating layer. In this case, for the platelet retention rate (the ratio of the number of platelets in blood after the blood circulation to the number of platelets in blood before the blood circulation; a value obtained by Equation 1 described in Examples), higher values indicate that thrombus is not being formed, and lower values indicate that thrombus is being formed. Therefore, the upper limit of the platelet retention rate is 100% (that is, in a state where no thrombus is formed). In the evaluation of the platelet retention rate, specific measurement methods and measurement conditions of the number of platelets may be adopted from those described in the Examples.

In the medical device disclosed here, the platelet retention rate by the evaluation using blood samples which has a heparin concentration of 0.2 units/mL is 80% or more, but is preferably 85% or more and more preferably 90% or more. Further, when the heparin concentration in the blood sample is 0.4 units/mL, the platelet retention rate is preferably 85% or more, more preferably 90% or more, and even more preferably 95% or more. Furthermore, when the heparin concentration in the blood sample is 1 unit/mL, it is preferred that the platelet retention rate is 100%.

The medical device, which includes a surface lubricating layer exhibiting the above properties, exhibits excellent antithrombotic qualities. Because the hydrophilic polymer (a) and the antithrombotic material (b) are each the same as those used in the production method, the detailed description thereof will be omitted.

Examples of the medical device according to the disclosure here include, for example, intracorporal implanted type artificial organs or treatment devices, extracorporeal circulating type artificial organs, catheters, guide wires, and the like. Specific examples thereof include artificial blood vessels, artificial trachea, or stent to be inserted or substituted into the blood vessel and the lumen, embedding type medical devices such as artificial skin and artificial pericardium, artificial organ systems such as artificial heart system, artificial lung system, artificial heart-lung system, artificial kidney systems, artificial liver system, and immune regulation system, or a catheter to be inserted or placed into the blood vessel, such as an indwelling needle, an IVH catheter, a catheter for administering a drug, a thermo dilution catheter, an angiographic catheter, a vasodilation catheter, a dilator or an introducer, or a guide wire for these catheters, a stylet or catheters to be inserted or placed into biological tissues other than the blood vessel, such as a stomach tube catheter, a nutrition catheter, a tube for cervical nutrition (ED), a urinary catheter, a guide urinary catheter, a balloon catheter, various suction catheters which includes an endotracheal suction catheter or a drainage catheter. In particular, the medical device according to the disclosure here may prevent thrombus from being formed on the surface of a medical device to prevent the thrombus from being scattered in the blood vessel, and thus is suitably used for treatments which are greatly affected by the scattering of thrombus in the living body. Specifically, various catheters used for the neuroendovascular therapy are suitably applied. Since the blood vessels are thin in the neuroendovascular therapy, the scattering of thrombus in the blood vessel is highly likely to be developed into cerebral infarction, and the like. For that reason, medical devices for neuroendovascular therapy are required to have lubricity for enhancing operability and antithrombotic qualities which prevent thrombus from being formed.

### EXAMPLE(S)

The effects of the inventive method and medical device disclosed here will be described by using the following Examples and Comparative Examples. However, the technical scope of the present invention is not limited to the following Examples.

### Synthesis Example 1

### Preparation of Hydrophilic Polymer

29.7 g of triethylene glycol was added dropwise to 72.3 g of adipic acid dichloride at 50° C., and then 4.5 g of methyl ethyl ketone was added to 22.5 g of oligoester obtained by removing hydrochloric acid under reduced pressure at 50° C. for 3 hours, and the resulting solution was added dropwise to a solution composed of 5 g of sodium hydroxide, 6.93 g of 31% hydrogen peroxide, 0.44 g of a surfactant dioctyl phosphate, and 120 g of water to perform a reaction at −5° C. for 20 minutes. The resulting product was washed repeatedly with water and methanol, and then dried to obtain a polyperoxide (PPO) containing a plurality of peroxide groups in the molecule thereof. Subsequently, 0.5 g of this PPO as a polymerization initiator, 9.5 g of glycidyl methacrylate (GMA), and 30 g of benzene as a solvent were polymerized while being stirred at 65° C. for 2 hours under a reduced pressure. The reaction product was reprecipitated in diethyl ether to obtain poly GMA (PPO-GMA) containing a peroxide group in the molecule.

Subsequently, 1 g of the resulting PPO-GMA as a polymerization initiator and 10 g of N,N-dimethyl acryl amide (DMAA) as a hydrophilic monomer were dissolved in 90 g of DMSO and heated at 70° C. for 8 hours to polymerize the monomer, thereby obtaining a polymer (hydrophilic polymer). The polymer (hydrophilic polymer) is a block copolymer (DMAA-GMA copolymer) having a poly GMA as a reactive site and a poly DMAA as a hydrophilic site having water swellability. Also, the composition (molar ratio) of the polymer (hydrophilic polymer) was analyzed with 1H-NMR, and it was found to have DMAA:GMA=11:1.

### Synthesis Example 2

### Preparation of Antithrombotic Material

9.95 g (48 mmol) of 2-acrylamido-2-methylpropanesulfonic acid (AMPS) and 0.43 g (6 mmol) of acrylic acid (AA) were dissolved in 100 mL of pure water, and put into a four-neck flask to carry out nitrogen bubbling in an oil bath at 50° C. for 1 hour, and then a solution of 0.146 g (1 mol %) of potassium persulfate (KPS) and 0.068 g (mole equivalent to KPS) of sodium sulfite dissolved in 1 mL of water and purged with nitrogen was added thereto. While continuously carrying out nitrogen bubbling, a solution obtained as described above was stirred (100 rpm) for 5 hours to carry out a polymerization at 50° C. After the polymerization reaction, the resulting aqueous solution was put into a dialysis membrane (fractionated molecular weight 12,000 to 14,000) and dialyzed with reverse osmosis water for 3 days or more to carry out purification. Further, thereafter, the solution was freeze-dried to obtain an AMPS-AA copolymer (antithrombotic material). The resulting polymer (antithrombotic material) was found to have a weight average molecular weight of 2,720,000, a number average molecular weight of 430,000 (Mw/Mn=6.2), and a composition ratio of AMPS:AA=8:1. The weight average molecular weight is a value measured by gel permeation chromatography (GPC) using pullulan as a standard material and water as a mobile phase. Also, the composition (molar ratio) of the polymer was analyzed by 1H-NMR.

### Example 1-1

### Concentration of Antithrombotic Material: 0.01 wt %

As a substrate, a soft polyvinyl chloride (PVC) tube having an inner diameter of 6 mm and an outer diameter of 9 mm was cut into a 45 cm length. A 5 wt % DMF solution of the hydrophilic polymer (DMAA-GMA copolymer) obtained in Synthesis Example 1 was prepared. Subsequently, a tube was filled with the solution to immerse the PVC (substrate) into a solution of the hydrophilic polymer at room temperature (25° C.), and after 5 seconds, the substrate was removed. Thereafter, the substrate was heated and dried in an oven set to 80° C. for 5 hours to form a coating layer having a thickness (film thickness after drying) of 3 μm.

A 0.01 wt % aqueous solution of the 2-acrylamido-2-methylpropanesulfonic acid (AMPS) acrylic acid copolymer (AMPS-AA copolymer) obtained in Synthesis Example 2 was prepared. Subsequently, a tube was filled with the aqueous solution and left to stand at room temperature (25° C.) for 10 minutes, the coating layer was immersed into the AMPS-AA solution, and then the filled solution was removed. Thereafter, the tube was heated and dried at 80° C. for 5 hours to form a surface lubricating layer. Thereafter, the tube was put into a mass cylinder filled with reverse osmosis water and left to stand for 24 hours or more, and an AMPS-AA copolymer, which was not fixed, was washed. In this case, the AMPS-AA copolymer fixed to the hydrophilic polymer was not released by washing with reverse osmosis water. Also, thereafter, the copolymer was left to stand and dried in an oven set to 50° C. for 12 hours or more to obtain Sample (1-1). In this case, the weight ratio of the antithrombotic material based on the total weight of the surface lubricating layer was 0.006 wt %. The weight ratio of the antithrombotic material based on the total weight of the surface lubricating layer was measured as follows (unless otherwise specifically described, the same applies thereafter).

First, (i) to (iii) were obtained by Equations (1) to (3) below.

(i) Amount of Hydrophilic Polymer Coated (Amount of Coating Layer Coated):

(Weight of substrate after coating and drying of the hydrophilic polymer)−(Weight of substrate before coating)=Amount of hydrophilic polymer coated (i) Equation (1)

(ii) Amount of Antithrombotic Material Solution Impregnated:

(Weight of substrate immediately after impregnation of the antithrombotic solution)−(Weight of substrate after coating and drying of the hydrophilic polymer)=Amount of antithrombotic material solution impregnated (ii) Equation (2)

(iii) Weight of Antithrombotic Material Included in Hydrophilic Polymer Coating Layer (Coating Layer)

(Amount of antithrombotic material solution impregnated (ii))×(Concentration of antithrombotic material solution (%)/100)=Weight of antithrombotic Material Included in Hydrophilic Polymer Coating Layer Equation (3)

Subsequently, (i) to (iii) obtained as described above were used to obtain the weight ratio of the antithrombotic material in the surface lubricating layer (that is, the weight ratio of the antithrombotic material (b) based on the total weight of the surface lubricating layer) from Equation (4) below.

The weight ratio (%) of the antithrombotic material in the surface lubricating layer: (iii)÷{(i)+(iii)}×100 Equation(4).

### Example 1-2

### Concentration of Antithrombotic Material: 0.05 wt %

Sample (1-2) was obtained in the same manner as in Example 1-1, except that the concentration of the aqueous solution of the AMPS-AA copolymer was changed into 0.05 wt %. In this case, the weight ratio of the antithrombotic material with respect to the total weight of the surface lubricating layer was 0.031 wt %.

### Comparative Example 1-1

### Concentration of Antithrombotic Material: 1 wt %

Comparative Sample (1-1) was obtained in the same manner as in Example 1-1, except that the concentration of the aqueous solution of the AMPS-AA copolymer was changed into 1 wt %. In this case, the weight ratio of the antithrombotic material based on the total weight of the surface lubricating layer was 0.63 wt %.

### Comparative Example 1-2

### Concentration of Antithrombotic Material: 0.1 wt %

Comparative Sample (1-2) was obtained in the same manner as in Example 1-1, except that the concentration of the aqueous solution of the AMPS-AA copolymer was changed into 0.1 wt %. In this case, the weight ratio of the antithrombotic material based on the total weight of the surface lubricating layer was 0.063 wt %.

### Comparative Example 1-3

### Concentration of Antithrombotic Material: 0 wt %

Comparative Sample (1-3) was obtained in the same manner as in Example 1-1, except that the treatment using an aqueous solution of the AMPS-AA copolymer was not carried out. In this case, the weight ratio of the antithrombotic material based on the total weight of the surface lubricating layer was 0 wt %.

### Comparative Example 1-4

### Concentration of Antithrombotic Material: 0.001 wt %, Simultaneous Application of Hydrophilic Polymer and Antithrombotic Material

As a substrate, a soft polyvinyl chloride (PVC) tube having an inner diameter of 6 mm and an outer diameter of 9 mm was cut into a 45 cm length. A DMF solution that contains 5 wt % of the hydrophilic polymer (DMAA-GMA) obtained in Synthesis Example 1 and 0.001 wt % of the 2-acrylamido-2-methylpropanesulfonic acid (AMPS) acrylic acid copolymer (AMPS-AA copolymer) obtained in Synthesis Example 2 was prepared. Subsequently, a tube was filled with the solution and left to stand at room temperature (25° C.) for 5 minutes, the substrate (tube) was immersed into the solution, and then the filled solution was removed. Thereafter, the substrate was heated and dried at 80° C. for 5 hours to form a surface lubricating layer having a thickness (dried film thickness) of 3 μm. Thereafter, the tube was put into a mass cylinder filled with reverse osmosis water and left to stand for 24 hours or more, and the hydrophilic polymer and an AMPS-AA copolymer, which were not fixed, were washed. Also, thereafter, the copolymer was left to stand and dried in an oven set to 50° C. for 12 hours or more to obtain a comparative sample (1-4). In this case, the weight ratio of the antithrombotic material based on the total weight of the surface lubricating layer was 0.02 wt %. However, the weight ratio of the antithrombotic material in this case was calculated based on the weight ratio of the hydrophilic polymer (DMAA-GMA) and the antithrombotic material (AMPS-AA copolymer) included in the DMF solution.

### Example 2-1

### Concentration of Antithrombotic Material: 0.01 wt %

As a substrate, a Nylon (registered trade name, hereinafter the same applies) elastomer sheet (manufactured by MSK Japan Ltd., Grilamid ELG6260) was cut into a size of 2 cm×6 cm. A 5 wt % DMF solution of the hydrophilic polymer (DMAA-GMA copolymer) obtained in Synthesis Example 1 was prepared. Subsequently, the Nylon elastomer sheet was immersed into the solution at room temperature (25° C.), and after 5 seconds, the sheet was taken out. Thereafter, the Nylon elastomer sheet was heated and dried in an oven set to 80° C. for 5 hours to form a coating layer having a thickness (film thickness after drying) of 2 μm.

A 0.01 wt % aqueous solution of the 2-acrylamido-2-methylpropanesulfonic acid (AMPS) acrylic acid copolymer (AMPS-AA copolymer) obtained in Synthesis Example 2 was prepared. Subsequently, the Nylon elastomer sheet was immersed into the aqueous solution and left to stand at room temperature (25° C.) for 10 minutes, and then the Nylon elastomer sheet was taken out, and heated and dried at 80° C. for 5 hours. Thereafter, the Nylon elastomer sheet was put into a case filled with reverse osmosis water and left to stand for 24 hours or more, and an AMPS-AA copolymer, which was not fixed, was washed. Also, thereafter, the copolymer was left to stand and dried for 12 hours or more in an oven set to 50° C. to obtain Sample (2-1). In this case, the weight ratio of the antithrombotic material based on the total weight of the surface lubricating layer was 0.006 wt %.

### Example 2-2

### Concentration of Antithrombotic Material: 0.05 wt %

Sample (2-2) was obtained in the same manner as in Example 2-1, except that the concentration of the aqueous solution of the AMPS-AA copolymer was changed into 0.05 wt %. In this case, the weight ratio of the antithrombotic material based on the total weight of the surface lubricating layer was 0.03 wt %.

### Comparative Example 2-1

### Concentration of Antithrombotic Material: 1 wt %

Comparative Sample (2-1) was obtained in the same manner as in Example 2-1, except that the concentration of the aqueous solution of the AMPS-AA copolymer was changed into 1 wt %. In this case, the weight ratio of the antithrombotic material based on the total weight of the surface lubricating layer was 0.63 wt %.

### Comparative Example 2-2

### Concentration of Antithrombotic Material: 0.1 wt %

A comparative sample (2-2) was obtained in the same manner as in Example 2-1, except that the concentration of the aqueous solution of the AMPS-AA copolymer was changed into 0.1 wt %. Note that, in this case, the weight ratio of the antithrombotic material based on the total weight of the surface lubricating layer was 0.06 wt %.

### Test Example 1

### Test of Antithrombotic Qualities (Platelet Retention Rate)

In order to evaluate the antithrombotic of the antithrombotic material under severe conditions in which thrombus was easily formed, the following test system was constructed and Samples (1-1) and (1-2) and Comparative Samples (1-1) to (1-4) were used to evaluate the antithrombotic qualities as follows.

A mixture of 0.56 mL of HEPAFLUSH 100A (heparin concentration 100 units/mL) and 8.0 mL of physiological saline was prepared in advance in a plastic-made test tube having a volume of 50 mL.

48 mL of fresh human blood was added thereto to prepare 56 mL of blood having a heparin concentration 1.0 units/mL. Herein, the fresh blood refers to blood collected from a normal healthy donor by whole blood transfusion, and blood collected within 30 minutes.

By the same method as described above, blood having a heparin concentration of 0.4 units/mL and 0.2 units/mL, respectively was prepared by changing only the amount of HEPAFLUSH 100 added.

The lumen of each sample (tube) obtained in the Examples and the Comparative Examples was filled with the blood prepared as described above. Thereafter, the tube (sample) filled with blood was kept in a loop shape, and the ends of the tube were connected with each other by a connector to provide the tube on a cylindrical rotation device. The tube was rotated at 40 rpm at room temperature (25° C.) for 2 hours to circulate blood. The blood after circulation was recovered, and for the sampled blood, the number of platelets was measured by using a blood count measuring apparatus (Sysmex).

As in Equation 1 below, the ratio of the number of platelets after circulation to the number of platelets before circulation was calculated as a platelet retention rate. For the platelet retention rate, high values indicate that thrombus has not been formed, and low values indicated that thrombus has been formed. The results are shown in Table 1, with the platelet retention rate identified by the percentage numbers in the three right-most columns.

[Math. 3]

Platelet retention rate (%)=(Number of platelets in blood 2 hours after blood circulation)/(Number of platelets in blood before blood circulation)×100 (Equation 1)

TABLE 1

| | Weight ratio of | | | |
|---|---|---|---|---|
| | antithrombotic material | | | |
| | based on total weight | Concentration of heparin | | |
| | of surface lubricating | in circulating blood | | |
| | layer (wt %) | 1 units/mL | 0.4 units/mL | 0.2 units/mL |
| Example 1-1 | 0.006 | 100% | 100% | 95% |
| (Concentration of | | | | |
| antithrombotic material: | | | | |
| 0.01%) | | | | |
| Example 1-2 | 0.031 | 100% | 100% | 98% |
| (Concentration of | | | | |
| antithrombotic material: | | | | |
| 0.05%) | | | | |
| Comparative Example 1-1 | 0.63 | 100% | 84% | 5.5% |
| (Concentration of | | | | |
| antithrombotic material: 1%) | | | | |
| Comparative Example 1-2 | 0.063 | 100% | 86% | 0.3% |
| (Concentration of | | | | |
| antithrombotic material: | | | | |
| 0.1%) | | | | |
| Comparative Example 1-3 | 0 | 100% | 88% | 0.4% |
| (Concentration of | | | | |
| antithrombotic material: 0%) | | | | |
| Comparative Example 1-4 | 0.02 | 100% | 88% | 0.4% |
| (Concentration of | | | | |
| antithrombotic material: | | | | |
| 0.001%) Simultaneous | | | | |
| application | | | | |

From Table 1, it can be seen that at a heparin concentration of 1 unit/mL, no difference in platelet retention rate was made in any sample, and at a heparin concentration 0.4 units/mL and 0.2 units/mL, a great difference was observed. In particular, under severe conditions in which thrombus is easily formed when the concentration of heparin in the circulating blood was 0.2 units/mL, a significant difference in platelet retention rate is made. Moreover, in the sample according to the disclosure here, it was revealed that the platelet retention rate was very high. These results indicate that a medical material having excellent antithrombotic qualities may be provided according to the disclosure here.

Also, in Comparative Examples 1-4, it was revealed that the weight ratio of the antithrombotic material based on the total weight of the surface lubricating layer was large, but sufficient antithrombotic qualities failed to be obtained. This indicates that not only the weight ratio of the antithrombotic material, but also the state of the antithrombotic material distributed in the surface lubricating layer greatly contribute to the antithrombotic qualities. That is, since the antithrombotic material was uniformly distributed in the surface lubricating layer in Comparative Example 1-4, whereas the antithrombotic material is greatly distributed on the surface of the surface lubricating layer (that is, a surface brought into contact with blood) in Examples 1-1 and 1-2, it is inferred that antithrombotic qualities were improving.

### Test Example 2

### Test of Antithrombotic Qualities (Surface Observation)

As in Test Example 1, after blood was circulated in the samples (tubes) manufactured in the Examples and the Comparative Examples, the surface in a tube was observed by an electron microscope (manufactured by Hitachi, Ltd., magnification 2,000 fold).

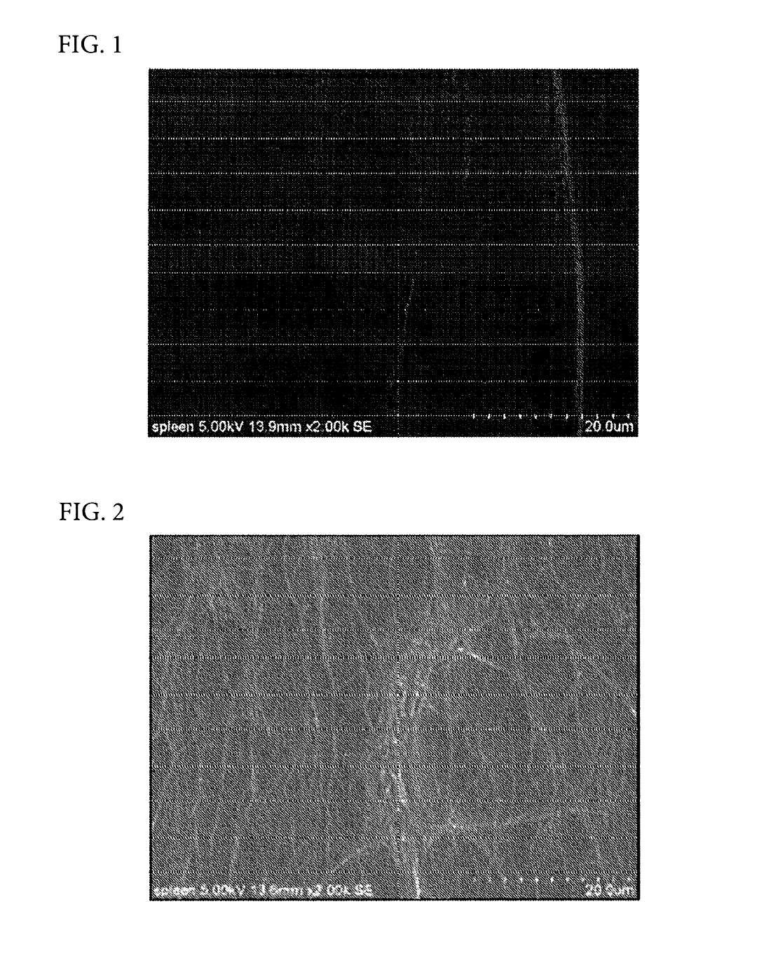

FIG. 1 is an enlarged photograph of the surface in the tube immediately after the antithrombotic test in Sample (1-1) manufactured in Example 1-1. FIG. 2 is an enlarged photograph of the surface in the tube immediately after the antithrombotic test in Comparative Sample (1-2) manufactured in Comparative Example 1-2. These enlarged photographs are those of the surface in the tube after blood having a heparin concentration of 0.2 units/mL was circulated. As a result, in the sample of Example 1-1 according to the disclosure here, the surface was smooth even after the circulation of blood and it is not acknowledged that thrombus was formed (FIG. 1). In addition, the same also applies to the samples manufactured in the other Examples. Meanwhile, it was confirmed that in the sample of Comparative Example 1-2, fibrous thrombus had been formed on the surface thereof (FIG. 2). In addition, the same also applies to the samples manufactured in the other Comparative Examples.

Therefore, as illustrated in Table 1 and FIGS. 1 and 2, it can be seen that the medical device according to the disclosure here exhibits excellent antithrombotic qualities even when being used under severe conditions in which thrombus is usually easily formed.

### Test Example 3

### Surface Lubricity Test

The samples (sheets) manufactured in Examples 2-1 and 2-2 and Comparative Examples 2-1 and 2-2 were immersed into physiological saline, and then rubbed by fingers. As a result, the surface of the substrate in any sample (sheet) had slippery lubricity. Furthermore, lubricity was maintained in any sample (sheet) even after being repeatedly rubbed by fingers 50 times.

As described above, from the results in Test Examples 1 to 3, it was revealed that the medical device according to the disclosure here also has excellent surface lubricity together with excellent antithrombotic qualities.

The detailed description above describes embodiments of a medical device and a method of producing a medical device representing examples of the inventive medical device and method of producing a medical device disclosed here. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for producing a medical device comprised of a surface lubricating layer on a surface of a substrate, in which the surface lubricating layer exhibits lubricity and antithrombotic qualities when being wet, the method comprising:
forming onto the substrate a coating layer that contains a hydrophilic polymer having at least one reactive functional group selected from the group consisting of an epoxy group, an acid chloride group, and an aldehyde group;
applying onto the coating layer an antithrombotic material solution that contains an antithrombotic material having a functional group that binds to the hydrophilic polymer to form the surface lubricating layer;
the antithrombotic material in the antithrombotic material solution being present in a concentration more than 0 wt % and less than 0.1 wt %.

2. The method for producing a medical device according to claim 1, wherein the antithrombotic material has a sulfonate group or a sulfate group.

3. The method for producing a medical device according to claim 2, wherein the antithrombotic material contains a repeating unit derived from a monomer selected from the group consisting of 2-(meth)acrylamido-2-methyl-propanesulfonic acid, vinyl sulfate, allyl sulfate, styrenesulfonic acid, sulfoethyl (meth)acrylate, and sulfopropyl (meth)acrylate, or salts thereof.

4. The method for producing a medical device according to claim 3, wherein the functional group configured to bind to the hydrophilic polymer is a carboxyl group, a hydroxyl group, or a thiol group.

5. The method for producing a medical device according to claim 4, wherein the hydrophilic polymer is obtained by copolymerizing a monomer having the reactive functional group with a hydrophilic monomer.

6. The method for producing a medical device according to claim 1, wherein the functional group of the antithrombotic material is a functional group which is crosslinkable with the reactive functional group of the hydrophilic polymer in the hydrophilic polymer of the coating layer.

7. The method for producing a medical device according to claim 1, further comprising subjecting the coating layer that contains the hydrophilic polymer to heat treatment after forming the coating layer on the substrate.

8. The method for producing a medical device according to claim 1, wherein the antithrombotic material contains a repeating unit derived from a monomer selected from the group consisting of 2-(meth)acrylamido-2-methyl-propanesulfonic acid, vinyl sulfate, allyl sulfate, styrenesulfonic acid, sulfoethyl (meth)acrylate, and sulfopropyl (meth)acrylate, or salts thereof.

9. The method for producing a medical device according to claim 1, wherein the functional group configured to bind to the hydrophilic polymer (a) is a carboxyl group, a hydroxyl group, or a thiol group.

10. The method for producing a medical device according to claim 1, wherein the hydrophilic polymer is obtained by copolymerizing a monomer having the reactive functional group with a hydrophilic monomer.

11. The method for producing a medical device according to claim 1, further comprising surface treating the substrate before forming the coating layer on the substrate.

12. The method for producing a medical device according to claim 1, wherein the functional group of the antithrombotic material is a negatively charged functional group.

13. The method for producing a medical device according to claim 1, wherein the applying of the antithrombotic material solution onto the coating layer is accomplished by immersing the substrate with the coating layer into the antithrombotic material solution.

14. The method for producing a medical device according to claim 1, further comprising modifying the surface lubricating layer by adding an other antithrombotic material.

15. A medical device comprising:
a surface lubricating layer on a surface of a substrate, the surface exhibiting lubricity and antithrombotic qualities when being wet;
the surface lubricating layer containing a hydrophilic polymer having at least one reactive functional group selected from the group consisting of an epoxy group, an acid chloride group, and an aldehyde group, and an antithrombotic material having a functional group that binds to the hydrophilic polymer;
the antithrombotic material is contained in an amount of more than 0 wt % and less than 0.06 wt % based on a total weight of the surface lubricating layer; and
when blood having a heparin concentration of 0.2 units/mL is brought into contact with the surface lubricating layer and circulated, a platelet retention rate determined by Equation 1 below is 80% or more.
Platelet retention rate (%)=(Number of platelets in blood 2 hours after blood circulation)/(Number of platelets in blood before blood circulation)×100 (Equation 1)

16. The medical device according to claim 15, wherein the antithrombotic material has a sulfonate group or a sulfate group.

17. The medical device according to claim 15, wherein the antithrombotic material contains a repeating unit derived from a monomer selected from the group consisting of 2-(meth)acrylamido-2-methyl-propanesulfonic acid, vinyl sulfate, allyl sulfate, styrenesulfonic acid, sulfoethyl (meth)acrylate, and sulfopropyl (meth)acrylate, or salts thereof.

18. The medical device according to claim 15, wherein the functional group configured to bind to the hydrophilic polymer is a carboxyl group, a hydroxyl group, or a thiol group.
